(19) United States
(12) Patent Application Publication
(10) Pub. No.: US 2009/0188800 A1
(43) Pub. Date: Jul. 30, 2009

(54) ENHANCING PHORETIC SEPARATION

(76) Inventors: Mirianas Chachisvilis, San Diego, CA (US); Osman Kibar, San Diego, CA (US); Eugene Tu, San Diego, CA (US)

Correspondence Address: FISH & RICHARDSON PC, P.O. BOX 1022, MINNEAPOLIS, MN, 55440-1022, US

(21) Appl. No.: 12/360,239

(22) Filed: Jan. 27, 2009

Related U.S. Application Data
(60) Provisional application No. 61/024,695, filed on Jan. 30, 2008.

(51) Int. Cl. B01D 57/02; G01N 33/00; G06F 15/00; B01D 43/00

(52) U.S. Cl. 204/547; 204/450; 204/157.15; 204/600; 73/866; 702/127; 209/155

(57) ABSTRACT

Among other things, a force is applied, at a first location in a medium and at a first time, to cause an object to move in a direction along the medium. At a later, second time, a force is applied at a second location, which is farther along the direction in which the object is moving, to cause the object to move an additional distance in the medium, when the force is no longer being applied at the first location. Both the distance traveled by the object and how long the object is subject to the force depend on a property of the object. At least one of the times and locations of applying the force is selected based on the property of the object.

This application is entitled to the benefit of the filing date of U.S. provisional patent application Ser. 61/024,695, filed on Jan. 30, 2008.

### BACKGROUND

This description relates to enhancing phoretic separation.

A variety of techniques are known for separating objects of interest (e.g., biomolecules, beads, cells, nanoparticles) on the basis of a property (e.g., mass, charge). For example, phoretic separation techniques (PSTs) apply an external force that causes objects of interest to move distances that are determined by values of a particular property of the objects. We use the term phoresis broadly to refer to any motion of objects induced by any gradient of their potential energy, which in turn is induced by any external force.

For example, electrophoresis (EP) applies a constant voltage to a set of proteins in a gel; each protein species acquires a different speed based on its charge, due to interactions with the electric field, and size, due to an opposing viscous drag force. As examples, dielectrophoresis (DEP) employs an oscillating electric field with a spatial gradient to separate objects based on their dielectric constant; thermophoresis (TP) applies a temperature gradient that preferentially moves objects based on their surface properties; optophoresis (OP) applies a traveling optical field profile to distinguish objects based on their refractive indices.

Applications of these PSTs may broadly include manipulating one type of object (e.g., transport or enrichment of DNA molecules in whole blood), separating one type of object from other types (e.g., collection or filtering of aerosol particles from a fluid), or separating different species of the same type of object from one another (e.g., separation or sorting of various protein species from a mix of proteins).

### SUMMARY

In general, in an aspect, a force is applied, at a first location in a medium and at a first time, to cause an object to move in a direction along the medium. At a later, second time, a force is applied at a second location, which is farther along the direction in which the object is moving, to cause the object to move an additional distance in the medium, when the force is no longer applied at the first location. Both the distance traveled by the object and how long the object is subject to the force depend on a property of the object. At least one of the times and locations of applying the force is selected based on the property of the object.

Implementations may include one or more of the following features. The force is effected by a gradient that enables phoretic motion of an object. The force is effected by an electric field gradient and enables electrophoresis. The force is effected by an oscillating electric field gradient and enables dielectrophoresis. The force is effected by a temperature gradient and enables thermophoresis. The force is effected by an optical field gradient and enables optophoresis. The force is effected by a magnetic field gradient that enables magnetophoresis. The gradient has a non-constant spatial and temporal profile. The non-constant spatio-temporal profile is generated by using intermediate spatial fabrication features along the direction of motion. The shape of the gradient profile comprises at least one of a Gaussian, a linear, a triangular, a square wave, or a sawtooth shape. The range of the gradient comprises at least one of a large range or a small range. The magnitude of the gradient comprises at least one of a large magnitude or a small magnitude. The force is swept across the medium in the direction in which the object is being moved. The force is swept across the medium more than once. A time period between successive sweeps is varied. A spatial profile of the sweeps is not constant. The force is swept at multiple locations across portions of the medium simultaneously. The distance between adjacent sweeps is varied. A spatial profile of the sweeps is not constant. The sweep magnitude is not constant along a second dimension that is different from the sweep direction. The force is swept at a non-constant speed. The sweep speed is accelerated. The sweep speed is decelerated. The sweep speed has a pre-defined temporal profile. The force is swept at a speed related to the fallout velocity of the object. A certain property of the object is measured based on the distance that the object travels. The force is swept along more than one dimension. At least one of electrophoresis, dielectrophoresis, optophoresis, magnetophoresis, thermophoresis, ultrasound, or other phoretic separation technique is performed. An additional gravitational, flow, electrical, electromagnetic, magnetic, or thermal force is applied. The object comprises a biomolecule. The object comprises a nanoparticle. The object comprises a microparticle. The object comprises a protein. The object comprises a DNA molecule. The object comprises an RNA molecule. The object comprises a virus. The object comprises a bacterium. The object comprises a cell. The object is separated from another object in the medium. The absence or presence of the object in the medium is detected. A certain parameter of the object is analyzed or measured. The object is pre-labeled before moving a distance. The object is post-labeled after moving a distance. The distance the object moves is non-linear with respect to a property of the object.

The parameter comprises mobility of the object. The status of the object is determined (e.g., phosphorylation, glycosylation, lipid modification, post-translational modification, methylation).

At least one result is used to optimize, adjust, and/or otherwise control another parameter. At least one result is used to quantify or otherwise determine a performance parameter of the method. At least one result is used to improve reproducibility. At least one result is used to improve resolution. At least one result is used to improve repeatability. Calibration is used to improve reproducibility. The method is implemented by a combination of software and hardware. The software is used for calibration, control, and/or analysis. The object is pre-labeled the object. The object is post-labeled. The distance the object moves is non-linear with respect to a property of the object.

In general, in an aspect, a medium is arranged to receive an object and to permit the object to move in a direction along the medium in response to a force, and (b) a source is arranged to apply a force at a first time and at a first location to cause the object to move in the direction and to apply a force at a later, second time and at a second location, which is farther along the direction, to cause the object to move an additional distance in the medium, when the force is no longer being applied at the first location. Both the distance traveled by the object and how long the object is subject to the force being dependent on a property of the object.

Implementations may include one or more of the following features. There is a container for the medium. The container includes a channel. The channel comprises a capillary. The container includes more than one channel. The container includes a separation area. The container includes a connection area. The container includes a detection area. The container includes a sample loading area. The separation area comprises electrodes. The electrodes are less than 1 μm wide. The electrodes are 1 μm to 10 μm wide. The electrodes are 10 μm to 100 μm wide. The electrodes are more than 100 μm wide. The electrodes have a center-to-center pitch of less than 1 μm wide. The electrodes have a center-to-center pitch of 1 μm to 10 μm. The electrodes have a center-to-center pitch of 10 μm to 100 μm. The electrodes have a center-to-center pitch of more than 100 μm wide. The electrodes comprise metal. The electrodes comprise semiconductor. The electrodes comprise conductive polymer. The electrodes are in contact with the separation area. The electrodes are not in contact with the separation area. The connection area comprises electrode contacts that are compatible with electronic connectors. The source comprises active circuitry to control the electrodes. There is an optical detector. There is a non-optical detector. There is an impedance detector. The sample is focused. The focusing is done by electrodes. The a discontinuous buffer system to do the focusing. The focusing is done electrokinetically. The container is reusable. The container is disposable. The container is single use. The apparatus of claim or in which the container is fabricated using photolithography. The container is fabricated using LIGA. The container is fabricated using printing. The container is fabricated using stamping. There is a power supply, a waveform generator, and an electrical connector. There are detection or analysis components. There is a graphical user interface. The detection or analysis components operate in real-time. There is more than one container. The separation area comprises a chemical matrix. The separation area comprises a-dimensional array. The separation area comprises a-dimensional array.

These and other aspects and features, and combinations of them, can be expressed as systems, methods, apparatus, means for performing functions, program products, and in other ways.

Other advantages and features will become apparent from the following description and the claims.

### DESCRIPTION

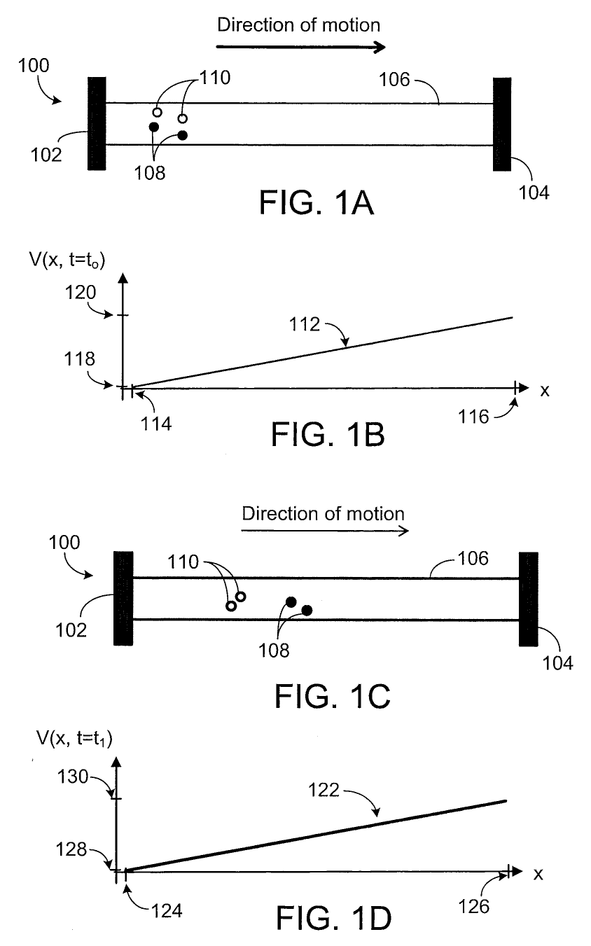

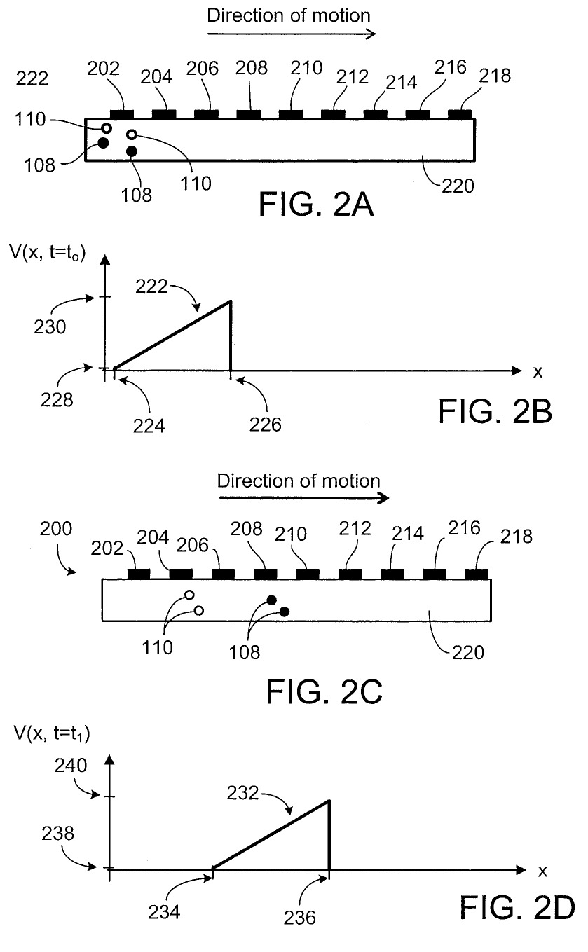

FIGS. 1A, 1C, 2A, and 2C are schematic sectional side views of electrophoresis.

FIGS. 1B, 1D, 2B, and 2D are graphs of applied voltage profiles.

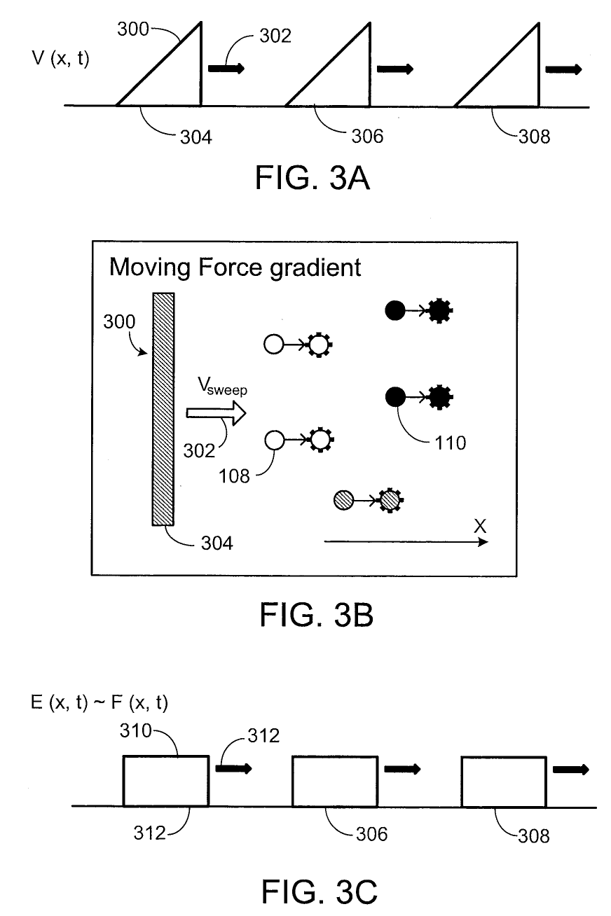

FIG. 3A is a graph of a swept voltage profile.

FIG. 3B is a schematic top view of a moving force gradient.

FIG. 3C is a graph of an applied electric field profile.

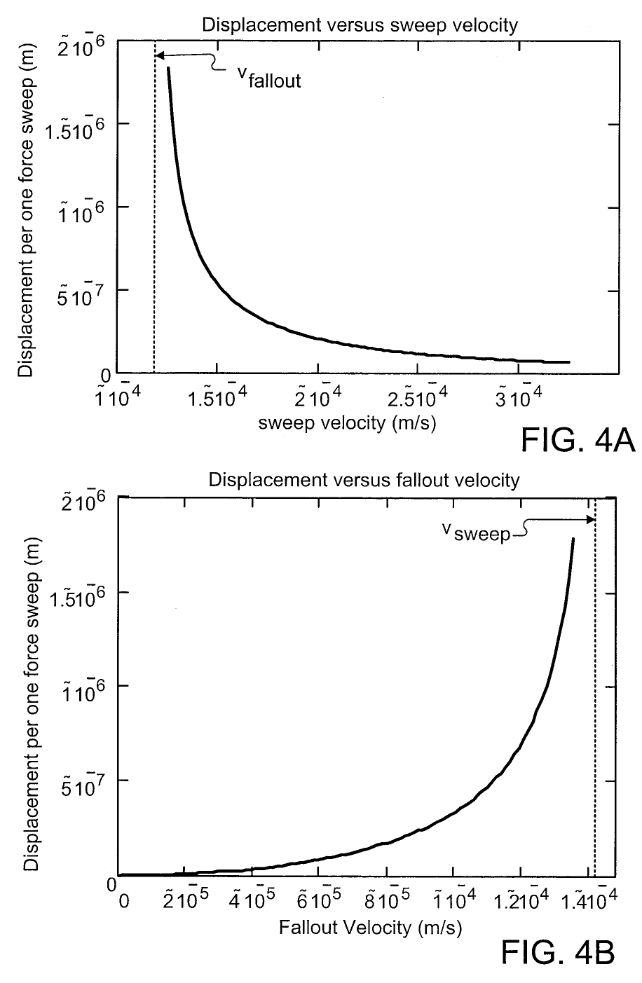

FIGS. 4A and 4B are graphs of distance traveled.

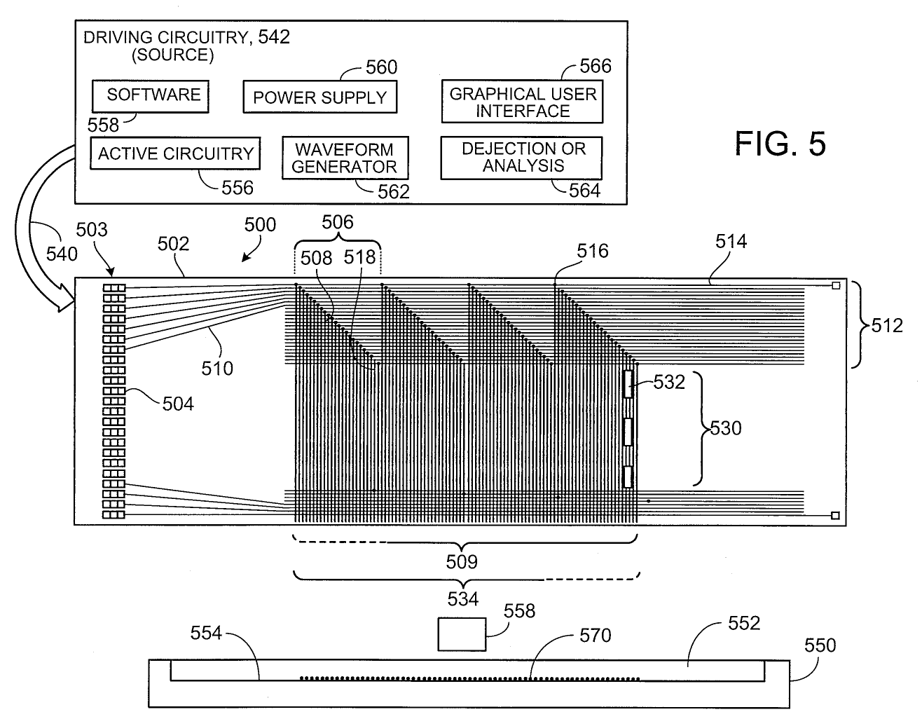

FIG. 5 is a top view of a separation device.

In known PSTs, because the distances that objects move are more or less linearly proportional to a property of the objects, relatively small differences in the values of the property for two objects produce a relatively small separation between the objects. For example, in EP, the distance traveled by an object depends linearly on a composite property of the object (in this case, a combination of its charge, size, environmental parameters, such as pH, and setup parameters, such as applied voltage and duration). For a given applied voltage, an instantaneous driving force and an opposing viscous drag force depend on the object's (e.g., protein's) composite property. Each protein will move at a corresponding velocity in the external electric field induced by the applied voltage. If two proteins have about a 1% difference in their composite properties, they will travel, on average, distances that are about 1% apart.

By modifying the spatial and temporal profiles of the applied force in a PST and tuning parameters of the applied force, objects having different composite property values within a certain range can be made to experience a nonlinear amplification of the total integrated applied force and to travel distances that are disproportionately different than the difference in their property values. Among other things, this nonlinear amplification may be used to physically separate and/or sort species of objects from one another with higher resolution.

The objects of interest may include, for example, proteins, enzymes, DNA, RNA, polymers, peptides, amino acids, other bio-molecules, molecules, viruses, cells, bacteria, beads, microparticles, or nanoparticles.

In some examples, EP (gel electrophoresis, in particular) is modified and used (alone or with other PSTs) to separate or to sort proteins (or other objects) with greater resolution.

Referring to FIG. 1A, a typical EP technique uses a setup 100 that applies a constant voltage between an electrode 102 and an electrode 104 at opposite ends of a gel 106. This voltage difference produces an electric field that imposes a spatially and temporally constant force on each protein (e.g., protein 108 belonging to one species and protein 110 belonging to another species) and moves positively-charged proteins towards the cathode (e.g., electrode 102) and negatively-charged proteins toward the anode (e.g., electrode 104).

Referring to FIG. 1B, a linear voltage profile 112, V(x,t), is shown that corresponds to the setup 100 (heterogeneities in the gel 106 are ignored). The voltage profile 112 is graphed at a time t₀; the end of setup 100 nearest electrode 102 has a voltage value 118 at position 114 and the end of setup 100 nearest electrode 104 has a voltage value 120 at position 116. This linear voltage profile 112 produces a uniform electric field across the gel 106. This electric field imposes constant (and possibly unequal) forces on proteins 108 and 110, regardless of their x positions.

Referring to FIG. 1C, the setup 100 is shown at a later time, t₁. Proteins 108 are shown closer to electrode 104 than proteins 110. In FIG. 1D, a linear voltage profile 122, V(x,t), is shown that corresponds to the setup 100 at time t₁ (heterogeneities in the gel 106 are again ignored). The end of setup 100 nearest electrode 102 has a voltage value 128 at position 124 and the end of setup 100 nearest electrode 104 has a voltage value 130 at position 126. It can be seen that voltage profile 112 is the same as voltage profile 122, specifically, in this example, the voltage value at position 114 at time t₀ equals the voltage value at position 124 at time t₁.

The distance traveled in a given time by an object (e.g., a charged particle) in the gel is determined by a net force that is the difference between the Coulomb force of the electric field and the viscous drag force, both acting on the object. For relatively small forces, this net force can be expressed as:

Fₙₑₜ=q*E−b*v, (1)

in which q is the net charge on the object, E, the magnitude of the electric field, b, a constant that depends on the properties of the gel and the dimensions of the object, and v, the velocity of the object.

Referring to FIG. 2A, in some examples of an approach described here, a setup 200 is used to produce a voltage profile that is not linear across the full set up and also not time-constant, e.g., a profile for which V(x,t) is a non-constant function of both x and t. In some examples, this type of voltage profile can be produced using a series of intermediate electrodes 202, 204, 206, 208, 210, 212, 214, 216, and 218 along the gel 220, rather than having electrodes only at the two ends. Proteins of a species 108 and proteins of a species 110 are shown in setup 200.

In some embodiments, the voltage profile is applied across the gel to produce an electric field that is non-uniform as a function of time. In FIG. 2B, for example, the voltage profile 222 is graphed at a time tₒ. At position 224, voltage profile 222 has a value 228 and at position 226, voltage profile 222 has a value 230. These values give voltage profile 222 the shape of a right triangle.

Referring to FIG. 2C, the setup 200 is shown at a later time, t₁. Proteins 108 are shown closer to electrode 218 than proteins 110. In FIG. 2D, a voltage profile 232 is shown that corresponds to the setup 200 at time t₁ (heterogeneities in the gel 220 are also ignored). At position 234, voltage profile 232 has a value 238 and at position 236, voltage profile 232 has a value 240. These values give voltage profile 232 the shape of a right triangle.

It can be seen that voltage profile 232 does not equal voltage profile 222, as the triangular waveform has been advanced spatially closer to electrode 218. Specifically, although both voltage profile 222 and voltage profile 232 are triangular waveforms of identical height (e.g., value 230 equals value 240), the position 236 at which voltage profile 232 equals value 240 has a greater x value than the position 226 at which voltage profile 222 equals value 230.

Referring to FIGS. 3A and 3B, a triangular waveform 300, in effect, moves in time (we sometimes say that it is swept), in one direction, at, e.g., a constant speed 302 (although in some examples, the speed would not need to be constant). FIG. 3A shows the waveform 300 at three different positions 304, 306, and 308 at three successive times. The right triangle shape is only for demonstrative purposes and is an idealized approximation of the actual voltage shape manifested in the gel. In FIG. 3B, the triangular waveform 300 traveling at speed 302 is shown schematically at location 304. Resulting displacements of proteins 108 and 110 are shown after a single sweep of the waveform 300 from left to right.

The corresponding instantaneous electric field profile 310, to which the force imposed on each object is proportional, is shown in FIG. 3C, at three positions 304, 306, and 308 at three successive times. The described voltage profile can be used to separate objects, for example, in a different way than by using typical EP. If the force profile 310 of FIG. 3C were moved to the right at a very slow speed 312 (i.e., the force is “swept”), viscous drag forces acting on the proteins would not be strong enough to prevent the proteins from moving along the gel at the same speed as the sweep velocity, and no separation of objects would be expected. In other words, all of the proteins would be swept along at the same velocity as the voltage profile is being swept.

As the sweep velocity is increased, however, the resulting viscous drag force on some proteins will equal or exceed the electric field force on these proteins, causing them to stop moving (which we sometimes call “falling out”). For a given protein, we call the sweep velocity at which the protein falls out, the “fallout velocity” of that protein. Different proteins may have different fallout velocities.

At a sweep velocity that is much higher than the fallout velocity of a protein, the viscous drag force becomes a quadratic function of velocity, and the net force becomes:

$${F}_{net} = q * E - \frac{ρ {v}^{2} A C}{2} , \qquad ( 2 )$$

in which ρ is the density of the gel, A, a reference area, and C, a drag coefficient. When subjected to this higher sweep velocity, a protein will move a finite distance during the time it is under the influence of the force profile that is sweeping past it. Proteins with higher fallout velocities will move slightly farther than proteins with lower fallout velocities because they are able to move at higher speeds during that period of interaction.

For example, assume two protein species (protein 108 and protein 110) have fallout velocities of 50 μm/s and 51 μm/s, respectively, for a given voltage profile. We sweep the whole voltage profile, V(x), at a velocity (e.g., 52 μm/s) slightly higher than the fallout velocities of both proteins. Because the fallout velocities of proteins 108 and 110 are close to the sweep velocity, proteins 108 and 110 will each move a significant distance when they are subject to the swept voltage profile.

Eventually, during the active period (when the proteins are subject to the force profile being swept), both proteins will fall out. Because of its higher fallout velocity, protein 110 will fall out later and therefore will be subjected to the influence of the sweeping force profile for more time than protein 108. This slightly longer exposure time will enable protein 110 to move farther (e.g., much farther) than protein 108 for each sweep, which magnifies the difference in distances traveled by the two proteins. By traveling farther and for a longer duration during each sweep, protein 110 is able to become more and more spatially separated from protein 108 with each sweep of the voltage profile, and, of course, as the sweep is repeated the separation becomes larger.

In this example, although the fallout velocities differ by only 2% (50 μm/s vs. 51 μm/s), the sweep velocity is close enough but above the fallout velocity of the faster protein (at least 52 μm/s in this example) to enable the two proteins to travel distances that differ by 20-30% of the total distance traveled (FIG. 4A, 4B). In other words, the separation resolution of the proteins can be amplified nonlinearly by (repeatedly) sweeping at a velocity that is slightly higher than the fallout velocity of the protein having the higher (highest) fallout velocity. As shown in FIG. 4A, the distance traveled by a protein becomes much greater when the sweep velocity is decreased toward the protein's fallout velocity. In FIG. 4B, the displacements of proteins with fallout velocities approaching the sweep velocity are much greater than for proteins having lower fallout velocities.

This technique (which we sometimes call the supersweep technique) may improve the performance-related parameters (e.g., resolution, speed, dynamic range, wider applicability) of any phoretic separation technique. And these improvements may be traded for setup-related parameters (e.g., reproducibility, cost) by modifying the system parameters (e.g., switching from a gel-based to a microfluidic-based system).

The technique described above may be varied, for example, as follows.

### Gradient Features

A gradient of the applied force may be generated by using intermediate electrodes and a spatio-temporal profile (i.e., function of both x and t). Or the gradient may be generated by using the current typical setup (with two electrodes on either end for EP) and vary the applied signal in time (i.e., function of only t), but also have a spatial non-constant environment (e.g., a wedged gel, such that the effective applied force is now also a function of x). Gradients could be generated in other ways and with other profiles.

The spatial feature that makes the force a function of x may be generated by a wide variety of techniques, including a heterogeneity of the geometry and/or the environment, e.g., a physical or chemical trap, a wedged gel, a microfluidic channel with a varying cross-section diameter.

The force gradient of interest may be generated by any one or a combination of a wide variety of physical methods. For example, a temperature gradient used in TP may be generated via electrical wires, laser beams, or acoustic/ultrasound pressure waves. If generated by electrodes, these electrodes may be in contact with the solution (or the gel) or isolated from the solution (or the gel). These electrodes may be of a different pitch, size, or shape. The force gradient may be effected by a magnetic field gradient that enables magnetophoresis.

The profile of the force gradient may be Gaussian, linear, triangular, square wave, sawtooth, or any other shape (geometric or arbitrarily defined).

The range of the applied gradient (e.g., the temperature range for TP or the voltage range for EP or DEP or the optical intensity range for OP) may be of any span, including from a small range to a large range. A range is considered small if the actual composite property of the object does not change (or changes very little) over the whole gradient range; similarly, the range is considered to be large if the relevant property of the object changes significantly over the complete range.

The magnitude of the applied gradient may be considered to be small or large. If small, it may be assumed that the actual relevant property of the object will not change much during operation. If the gradient magnitude is large, then the targeted property of the object will cover a finite range during operation.

### Sweep Parameters

At any one time, only one sweep may be performed over the media (e.g., gel, microfluidic channel, glass slide), or multiple sweeps may be moving at the same time or a combination of those approaches may be used, for example, at different times. Individual sweeps of the multiple-sweep approach may be equidistant from one another along the gel or may have arbitrarily-defined spacing between them. Similarly, these sweeps may be traveling at the same speed or at different speeds. The sweeps may have the same duty cycle and/or the shape, or they may have different duty cycles and/or shapes. The sweep magnitude may be increasing or decreasing over a second dimension (i.e., the magnitude may not be co-linear with the sweep direction).

Each sweep may be traveling at the same speed or at different speeds. The speed of each sweep may be constant, accelerating, decelerating, or may have an arbitrarily defined temporal profile (e.g., stop-and-go jerking motion). These sweeps may be traveling in the forward, backward, or in a combination thereof.

Sweep velocity may be tuned to the fallout velocity of a single object, and it may be set at well-below, slightly-below, equal to, slightly-above, or well-above that of the fallout velocity. The sweep velocity may also be adjusted with respect to a range of fallout velocities, in which it may cover a portion of such range, exactly cover the whole range, cover more than the range, be centered in the middle of the range, or be centered at a point other than the middle of the range (e.g., tuned to one particular species among many others).

Both a coarse separation and a fine separation may be achieved in the same setup, in which first the sweep velocity is increased (i.e., accelerated) as the sweep travels from one end to the other. This first run will cause the slower proteins to fall out early on and the faster ones to fall out at a farther distance from the start line. Then, a second sweep may be performed in the orthogonal direction to the first, in which the sweep velocity along each line is tuned to slightly above the corresponding fallout velocity at that line. For example, a sweep in the x-direction may be performed, starting with a sweep velocity of 30 μm/s at the start line and accelerating to 70 μm/s by the end of the sweep. As a result, those proteins with a fallout velocity of approximately 35 μm/s will drop out early on (e.g., at x=x1), and those with a fallout velocity of approximately 65 μm/s will travel a greater distance before falling out (e.g., at x=x2). Then, a second sweep may be performed in the y-direction, with a sweep starting at x1 equaling 36 μm/s and a second at x2 equaling 66 μm/s.

A form of iso-focusing (i.e. objects stop moving when their property matches that of a specific point along the x-axis in a similar fashion as iso-electric focusing in gel electrophoresis) may be performed by applying a gradient that is spatially and/or temporally asymmetric.

Certain object properties may be monitored, detected, or quantified based on the actual distance traveled by a particular species. The monitoring, detection, and quantification can be achieved by a variety of mechanisms and at a variety of locations and times.

The geometry of the separation medium need not be planar.

A wide variety of these features of the parameters of sweeps and combinations of them may be used.

### Environment/Setup/Geometry Related Variations

The medium and the sample may be held in a container.

The sweep may be performed in 1-, 2-, or 3-dimensions. Multiple sweeps may be run in the same container, or sequentially cascaded in more than one container.

If the sweep is being applied to objects in flow, the axis of the sweep may be oriented parallel to or perpendicular to or at an angle to the direction of flow.

The container may have one or more channels or no channels along which the motion occurs.

Certain environmental parameters may be manipulated (spatially or temporally), e.g., salt concentration, pH, viscosity, solvent/gel type, ionic strength, ambient temperature or pressure.

Separation and/or monitoring may be carried at as a time-of-flight process (i.e., finite duration for applied force) or as a steady-state equilibrium process (i.e., force may be applied indefinitely, and the objects will focus at a particular point after a sufficient time has passed).

Objects may be separated based on a single run, or in multiple steps (e.g., first a coarse separation, followed by a finer separation, etc.).

The supersweep technique may be used in tandem with other phoretic forces (e.g., with EP, DEP, TP, OP, flow, ultrasound).

This technique may also be combined in the same container with a counter-force of different origins (e.g., gravitational, flow, electrical, electromagnetic, magnetic, thermal).

### Functions/Applications

The various objects that the technique may be applicable to include, but are not limited to proteins, DNA, RNA, polymers, peptides, amino acids, enzymes, other bio-molecules, molecules, viruses, cells, bacteria, beads, microparticles, or nanoparticles.

The technique may be applied to separate, sort, detect the presence of or absence of various objects; and/or to analyze certain parameters of various objects (e.g. mobility, diffusion coefficient, and/or charge of the object); and/or to determine the status of various objects (e.g. existence or degree of phosphorylation, glycosylation, lipid modification or post-translational modification of proteins, or methylation of DNA). Such functions (e.g., separation, detection, analysis) may also be carried out based on post-translational-modifications (PTMs) of certain objects, especially of bio-molecules (e.g., epitope separation, or mutation scanning). Such functions may also be applied towards the fluctuations of particular objects (e.g., targeting second or third moments of protein fluctuations). One or more results of such functions can be used to optimize, adjust, and/or control (using feedback) other parameters (e.g., amplitude profile of voltage, sweep profile). One or more results can also be used to determine and/or quantify performance (e.g. determine the degree of separation and the number, amount and purity of the separated objects).

For example, one can measure the spatial distribution of the objects (i.e., their spatial spread and broadening) as a function of time, in a regime in which the objects are initially focused toward one electrode and then released to diffuse freely. The resulting spatial distribution of the objects can then be fitted to solutions of diffusion and drift equations, Gaussian distributions and other spatial distributions, and spatial distribution parameters of the object can be quantified and the mobility of the object determined. The result can then be compared to the spatial mobility dependence of a reference object to improve reproducibility. The result can also be used to optimize and/or adjust various control parameters to eliminate the spatial dependence of mobility, and thereby, to improve resolution and repeatability of the technique.

The technique may be employed in a qualitative or in a quantitative manner.

The technique may be employed to improve the reproducibility of the device and/or the experiment, e.g. by calibration using a known object's motion profile before, during, or after an experiment. Similar calibration techniques may also be employed to improve the reliability of the device and/or the results.

Various types of software may be used with this technique. For example, software can be used to calibrate the instrument, a consumable used during operation, or a particular experiment or run. The calibration can be done before, during, or after the experiment or run (i.e., pre-, in situ, or post-experiment, respectively) or a combination of them. The calibration can also be used for control purposes, e.g., to manipulate the sweep profile and parameters, to control and change the environmental parameters, or to optimize performance, or a combination of them. Software can also be used for analysis purposes, such as to predict performance, results and/or applicability, or to analyze (pre- and/or post-experiment) results or performance (e.g., deconvolution of data), or a combination of them. Although we use the word experiment sometimes in our discussion, we mean that word to apply also to pilot, commercial, productive, or other operations and techniques, not merely to ones that are experimental or hypothetical or at a laboratory scale.

Certain other techniques may be used to first reduce the sample complexity (i.e., sample preparation, such as using poly(T) to enrich for mRNA or lectins to select for protein glycosylation).

The technique may be combined with a post-detection technique (e.g., silver staining, mass spec, fluorescence detection).

The objects of interest may also be pre-labeled prior to separation, or they may be post-labeled after separation.

Various functions, algorithms, architecture, and/or concepts used in signal theory or information theory, and combinations of them, can be implemented as part of the techniques we have described (in hardware or in software or both). These implementations may include Fourier transforms and frequency domain operation, filtering, modulation, sampling, Laplace transforms, z-transforms, feedback systems, and others. These implementations may be employed to improve performance, such as noise reduction, better reproducibility, reliability, repeatability, and/or resolution, and combinations of them.

Motion of objects can be digitized, discretized, and/or quantized. Using variations of this technique, objects can be separated in discrete steps, where these steps can be binary or multi-valued, purely digital or a combination of digital and analog.

### Apparatus Related Variations:

Instrument: The container may have a separation area, a connection area, a detection area, and a sample loading area, or a combination of them. The container may have channels. It may be a capillary. It may consist of an array. Or a combination of them. The electrodes in the separation area may be less than 1 um wide, 1 to 10 um wide, 10 to 100 um wide, or more than 100 um wide, for example. The electrodes in the separation area may have a center-to-center pitch of less than 1 um, 1 to 10 um, 10 to 100 um, or more than 100 um, for example. The electrodes in the separation area may be metal (e.g., aluminum, gold, indium-tin-oxide), semiconductor (e.g., polysilicon), or conductive polymers, or a combination of them. There may be at least 3 electrodes in the separation area. There may be an electrode in contact with the top of the separation area to cause motion in the z dimension. The electrodes may be positioned above the substrate, or positioned flush or embedded in the substrate or a combination of them. External electrodes may be used (e.g., to apply high voltage across the separation area). The connection area of the container may have electrode contacts compatible with electronic connectors (e.g., edge connectors, pogo pins, z-axis elastomeric, or other surface connectors). Active circuitry may be used to control the voltage on the electrodes. The separation process may be detected optically or non-optically (e.g., using impedance effects) or a combination of them. The sample loading area may contain wells to apply sample volume. The sample may be focused using supersweep electrodes, in a continuous or discontinuous buffer system, or by electrokinetic means.

Consumables: The supersweep container may be reusable, e.g., washable between sample injections, or the container can be a single-use disposable (e.g., a consumable, a cartridge), or a combination of the two. In the single-use case, the ability to make consistent and reliable electrical contact between the container and the instrument is important. In some examples, the container has planar electrodes that are inserted into a connector, similar to edge electrodes and connectors found in PCMCIA cards and other electronics. Other topside connectors such as low/zero insertion force (zif), pogo pins, or z-axis elastomers, or a combination of them, may also be used. The latter connectors have greater alignment tolerances and may provide more reliable connections.

Fabrication process: The fabrication process can be selected based on the feature size required. For high resolution assays, smaller feature sizes suggest a use of photolithographic or LIGA type (lithography, electroplating, and molding) methods. Larger feature sizes allow methods such as printing or stamping, which are more cost effective. Suitable substrate materials include glass, fused silica, or polymers (e.g. SU-8, polyimide). The electrode and insulating layers can be patterned by deposition processes or by laminating alternate layers of conductors and dielectrics, or a combination of them. These layers can be rigid or flexible. The electrode material may be gold, platinum, indium-tin-oxide, graphite, conductive paste, or inks, or a combination of them. Semiconductor materials can also be used.

Optimizations: A shown in FIG. 5, the basic hardware required to implement the supersweep technique can include a power supply 560, a waveform generator 562, and a way to make electrical connection 540 to the sample container (which in FIG. 5 is fabricated on a substrate, holds the medium, and defines one or more channels for the moving objects). Enhancements to the instrumentation may include detection and analysis components and circuitry, and a GUI software interface 566. Real-time detection 564 allows for optimization of run parameters in-situ. The instrument may run multiple containers using different separation assays in parallel. The separation area of the container may contain a chemical matrix to aid in separation and/or to minimize diffusion (and thus, improve resolution). The container can be filled with (or contain) crosslinked polymers (e.g. polyacrylamide), thermoset polymers (e.g. agarose), or high molecular weight linear polymers (e.g. hydroxypropylcellulose). Physical properties such as pore size, viscosity, hydrophobicity, and charge can be optimized for different sample types.

Usage: The sample loading area of the container may contain wells to manually load the samples, similar to gel electrophoresis. Supersweep is compatible with some techniques used to focus the sample and increase resolution, such as sample stacking, gradient gels, and discontinuous buffer systems. The supersweep electrodes can be used to concentrate the sample(s) (e.g., compacting). The technique can be incorporated into a microfluidic or capillary device, which electrokinetically focuses the sample and then injects the sample into the supersweep separation area. This approach can be scaled into a 2- or 3-dimensional array for high throughput.

FIG. 5 depicts a device 500 fabricated on a glass substrate 502. At one end of the substrate a connection area 503 includes a row of pads 504 and a series of (in this case) four groups 506 (together situated in a separation/detection area 509) of evenly spaced gold electrodes 508 formed across the substrate. In the example, there are 24 electrodes of successively shorter lengths in each group. Connections 540 from driving circuitry 542 (not shown) to the electrodes are made through the pads and connection lines 510 that run from the pads to two sets 512 of parallel bus lines 514 (only some of the connection lines to the bus lines are shown in the figure).

In this example, the upper ends of corresponding electrodes of all the groups are generally connected in common to the same bus line by connections 516. The lower ends of the electrodes are generally left unconnected. Exceptions are that for some electrodes 518, the upper end is not connected to one of the upper buses but the lower end is connected to one of the lower buses for separation, sensing or detection purposes. The connections of the electrodes to buses is arranged to achieve one or more of the operational effects discussed earlier.

In addition to the connection and separation areas, there is a sample loading area 530 that includes sample loading wells 532, and a detection area 534. In the example of FIG. 5, the sample is loaded at the sample loading wells, the force is swept from right to left, and the detection is done at the left. The boundary between separation and detection areas is arbitrary. Both areas overlap the electrodes. The separation area begins at the sample loading area and extends left. When it is determined that enough separation has been achieved, the remaining area of the electrodes (from that point leftward) can be used for detection. Alternatively, the detection area can be substantially overlapping with the separation area or it can be a region or window within the separation area.

The lower portion of FIG. 5 schematically illustrates a side section of a container 550 that holds a medium 552 in which the objects move in one or more channels 554. The electrodes 570 can be formed on the upper surface of the container. An optical (or other) separation detector 558 is arranged to detect separation in the separation region of the device.

Other implementations are also within the scope of the following claims.

What is claimed is:

1. A method comprising
at a first location in a medium and at a first time, applying a force to cause an object to move in a direction along the medium;
at a later, second time, applying a force at a second location, which is farther along the direction in which the object is moving, to cause the object to move an additional distance in the medium, when the force is no longer being applied at the first location;
both the distance traveled by the object and how long the object is subject to the force being dependent on a property of the object;
at least one of the times and locations of applying the force being selected based on the property of the object.

2. The method of claim 1 in which the force is effected by a gradient that enables phoretic motion of an object.

3. The method of claim 2 in which the force is effected by an electric field gradient and enables electrophoresis.

4. The method of claim 2 in which the force is effected by an oscillating electric field gradient and enables dielectrophoresis.

5. The method of claim 2 in which the force is effected by a temperature gradient and enables thermophoresis.

6. The method of claim 2 in which the force is effected by an optical field gradient and enables optophoresis.

7. The method of claim 2 in which the force is effected by a magnetic field gradient that enables magnetophoresis.

8. The method of claim 2 in which the gradient has a non-constant spatial and temporal profile.

9. The method of claim 8 in which the non-constant spatio-temporal profile is generated by using intermediate spatial fabrication features along the direction of motion.

10. The method of claim 8 in which the shape of the gradient profile comprises at least one of a Gaussian, a linear, a triangular, a square wave, or a sawtooth shape.

11. The method of claim 2 in which the range of the gradient comprises at least one of a large range or a small range.

12. The method of claim 2 in which the magnitude of the gradient comprises at least one of a large magnitude or a small magnitude.

13. The method of claim 1 in which the force is swept across the medium in the direction in which the object is being moved.

14. The method of claim 13 in which the force is swept across the medium more than once.

15. The method of claim 14 in which a time period between successive sweeps is varied.

16. The method of claim 14 in which a spatial profile of the sweeps is not constant.

17. The method of claim 13 in which the force is swept at multiple locations across portions of the medium simultaneously.

18. The method of claim 17 in which a distance between adjacent sweeps is varied.

19. The method of claim 17 in which a spatial profile of the sweeps is not constant.

20. The method of claim 13 in which a magnitude of the sweep is not constant along a second dimension that is different from the sweep direction.

21. The method of claim 13 in which the force is swept at a non-constant speed.

22. The method of claim 21 in which the sweep speed is accelerated.

23. The method of claim 21 in which the sweep speed is decelerated.

24. The method of claim 21 in which the sweep speed has a pre-defined temporal profile.

25. The method of claim 13 in which the force is swept at a speed related to a fallout velocity of the object.

26. The method of claim 1 in which a certain property of the object is measured based on a distance that the object travels.

27. The method of claim 13 in which the force is swept along more than one dimension.

28. The method of claim 1 also including performing at least one of electrophoresis, dielectrophoresis, optophoresis, magnetophoresis, thermophoresis, ultrasound, or other phoretic separation technique.

29. The method of claim 1 also including applying an additional gravitational, flow, electrical, electromagnetic, magnetic, or thermal force.

30. The method of claim 1 in which the object comprises a biomolecule.

31. The method of claim 1 in which the object comprises a nanoparticle.

32. The method of claim 1 in which the object comprises a microparticle.

33. The method of claim 1 in which the object comprises a protein.

34. The method of claim 1 in which the object comprises a DNA molecule.

35. The method of claim 1 in which the object comprises an RNA molecule.

36. The method of claim 1 in which the object comprises a virus.

37. The method of claim 1 in which the object comprises a bacterium.

38. The method of claim 1 in which the objects comprises a cell.

39. The method of claim 1 also including separating the object from another object in the medium.

40. The method of claim 1 also including detecting the absence or presence of the object in the medium.

41. The method of claim 1 also including analyzing or measuring a certain parameter of the object.

42. The method of claim 41 in which the parameter comprises mobility of the object.

43. The method of claim 1 also including determining the status of the object.

44. The method of claim 43 in which the status comprises phosphorylation.

45. The method of claim 43 in which the status comprises glycosylation.

46. The method of claim 43 in which the status comprises lipid modification.

47. The method of claim 43 in which the status comprises post-translational modification.

48. The method of claim 43 in which the status comprises methylation.

49. The method of claim 39, 40, 41 or 43, in which at least one result is used to optimize, adjust, and/or otherwise control another parameter.

50. The method of claim 39, 40, 41 or 43, in which at least one result is used to quantify or otherwise determine a performance parameter of the method.

51. The method of claim 39, 40, 41 or 43, in which at least one result is used to improve reproducibility.

52. The method of claim 39, 40, 41 or 43, in which at least one result is used to improve resolution.

53. The method of claim 39, 40, 41 or 43, in which at least one result is used to improve repeatability.

54. The method of claim 1 in which calibration is used to improve reproducibility.

55. The method of claim 1 implemented by a combination of software and hardware.

56. The method of claim 55 in which software is used for calibration.

57. The method of claim 41 in which software is used for control.

58. The method of claim 41 in which software is used for analysis.

59. The method of claim 1 also comprising pre-labeling the object.

60. The method of claim 1 also comprising post-labeling the object.

61. The method of claim 1 in which a distance the object moves is non-linear with respect to a property of the object.

62. An apparatus comprising
a medium arranged to receive an object and to permit the object to move in a direction along the medium in response to a force,
a source arranged to apply a force at a first time and at a first location to cause the object to move in the direction and to apply a force at a later, second time and at a second location, which is farther along the direction, to cause the object to move an additional distance in the medium, when the force is no longer being applied at the first location;
both the distance traveled by the object and how long the object is subject to the force being dependent on a property of the object.

63. The apparatus of claim 62 also comprising a container for the medium.

64. The apparatus of claim 63 in which the container includes a channel.

65. The apparatus of claim 63 in which the channel comprises a capillary.

66. The apparatus of claim 63 in which the container includes more than one channel.

67. The apparatus of claim 63 in which the container includes a separation area.

68. The apparatus of claim 63 in which the container includes a connection area.

69. The apparatus of claim 63 in which the container includes a detection area.

70. The apparatus of claim 63 in which the container includes a sample loading area.

71. The apparatus of claim 67 in which the separation area comprises electrodes.

72. The apparatus of claim 71 in which the electrodes are less than 1 μm wide.

73. The apparatus of claim 71 in which the electrodes are 1 μm to 10 μm wide.

74. The apparatus of claim 71 in which the electrodes are 10 μm to 100 μm wide.

75. The apparatus of claim 71 in which the electrodes are more than 100 μm wide.

76. The apparatus of claim 71 in which the electrodes have a center-to-center pitch of less than 1 μm wide.

77. The apparatus of claim 71 in which the electrodes have a center-to-center pitch of 1 μm to 10 μm.

78. The apparatus of claim 71 in which the electrodes have a center-to-center pitch of 10 μm to 100 μm.

79. The apparatus of claim 71 in which the electrodes have a center-to-center pitch of more than 100 μm wide.

80. The apparatus of claim 71 in which the electrodes comprise metal.

81. The apparatus of claim 71 in which the electrodes comprise semiconductor.

82. The apparatus of claim 71 in which the electrodes comprise conductive polymer.

83. The apparatus of claim 71 in which the electrodes are in contact with the separation area.

84. The apparatus of claim 71 in which the electrodes are not in contact with the separation area.

85. The apparatus of claim 68 in which the connection area comprises electrode contacts that are compatible with electronic connectors.

86. The apparatus of claim 62 in which the source comprises active circuitry to control the electrodes.

87. The apparatus of claim 62 also comprising an optical detector.

88. The apparatus of claim 62 also comprising a non-optical detector.

89. The apparatus of claim 62 also comprising an impedance detector.

90. The apparatus of claim 62 in which a sample is focused.

91. The apparatus of claim 90 in which the focusing is done by electrodes.

92. The apparatus of claim 90 also comprising a discontinuous buffer system to do the focusing.

93. The apparatus of claim 90 in which the focusing is done electrokinetically.

94. The apparatus of claim 63 in which the container is reusable.

95. The apparatus of claim 63 in which the container is disposable.

96. The apparatus of claim 95 in which the container is single use.

97. The apparatus of claim 94 or 95 in which the container is fabricated using photolithography.

98. The apparatus of claim 63 in which the container is fabricated using LIGA.

99. The apparatus of claim 63 in which the container is fabricated using printing.

100. The apparatus of claim 63 in which the container is fabricated using stamping.

101. The apparatus of claim 62 also comprising a power supply, a waveform generator, and an electrical connector.

102. The apparatus of claim 101 also comprising detection or analysis components.

103. The apparatus of claim 101 also comprising a graphical user interface.

104. The apparatus of claim 102 in which the detection or analysis components operate in real-time.

105. The apparatus of claim 63 in which there is more than one container.

106. The apparatus of claim 67 in which the separation area comprises a chemical matrix.

107. The apparatus of claim 67 in which the separation area comprises a 2-dimensional array.

108. The apparatus of claim 67 in which the separation area comprises a 3-dimensional array.
